(19) United States
(12) Patent Application Publication
(10) Pub. No.: US 2009/0023799 A1
(43) Pub. Date: Jan. 22, 2009

(54) CRYSTALS OF (2-AMINO-4,5,6,7-TETRAHYDROBENZO[b]THIEN-3-YL)(4-CHLOROPHENYL)METHANONE

(76) Inventors: George Laurence, Lawrence, KS (US); Allan R. Moorman, Durham, NC (US); Aeri Park, West Lafayette, IN (US)

Correspondence Address: KING PHARMACEUTICALS, INC., 400 CROSSING BOULEVARD, BRIDGEWATER, NJ, 08807, US

(21) Appl. No.: 12/175,711

(22) Filed: Jul. 18, 2008

Related U.S. Application Data
(60) Provisional application No. 60/950,885, filed on Jul. 20, 2007.
(60) Provisional application No. 60/968,747, filed on Aug. 29, 2007.

(51) Int. Cl. A61K 31/381; C07D 333/66; A61P 25/00

(52) U.S. Cl. 514/443; 549/57

(57) ABSTRACT

The present invention provides crystal forms of (2-amino-4,5,6,7-tetrahydrobenzo[b]thiophen-3-yl)(4-chlorophenyl)methanone of the formula

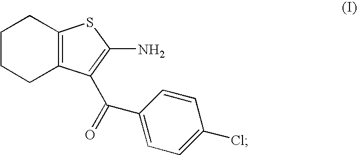

processes for the production of such crystal forms; pharmaceutical compositions comprising such crystal forms; and methods of treating diseases or conditions modulated by the adenosine A₁ receptor, in particular neuropathic pain, in a mammal in need thereof, by employing such crystal forms, or pharmaceutical compositions comprising such.

This application claims the benefit of U.S. Provisional Application No. 60/950,885 filed Jul. 20, 2007 and U.S. Provisional Application No. 60/968,747 filed Aug. 29, 2007, both of which are incorporated herein by reference in their entirety.

### FIELD OF INVENTION

The present invention provides crystal forms of (2-amino-4,5,6,7-tetrahydrobenzo[b]thiophen-3-yl)(4-chlorophenyl)methanone; processes for the production of such crystal forms; pharmaceutical compositions comprising such crystal forms; and methods of treating diseases or conditions modulated by the adenosine A₁ receptor by employing such crystal forms, or pharmaceutical compositions comprising such.

### BACKGROUND OF THE INVENTION

Adenosine is an endogenous nucleoside present in all cell types of the body. It is endogenously formed and released into the extracellular space under physiological and pathophysiological conditions characterized by an increased oxygen demand/supply ratio. This means that the formation of adenosine is accelerated in conditions with increased high energy phosphate degradation. The biological actions of adenosine are mediated through specific adenosine receptors located on the cell surface of various cell types, including nerves. The hyper-reactive nerves increase adenosine release due to an increase in metabolic activity.

Adenosine A₁ receptors are widely distributed in most species and mediate diverse biological effects. The following examples are intended to show the diversity of the presence of A₁ receptors rather than a comprehensive listing of all such receptors. A₁ receptors are particularly ubiquitous within the central nervous system (CNS), with high levels being expressed in the cerebral cortex, hippocampus, cerebellum, thalamus, brain stem, and spinal cord. Immuno-histochemical analysis using polyclonal antisera generated against rat and human adenosine A₁ receptors has identified different labeling densities of individual cells and their processes in selected regions of the brain. Adenosine A₁ receptor mRNA is widely distributed in peripheral tissues such as the vas deferens, testis, white adipose tissue, stomach, spleen, pituitary, adrenal, heart, aorta, liver, eye and bladder. Only very low levels of A₁ receptors are thought to be present in lung, kidney, and small intestine.

Adenosine has been proposed as a treatment for pain states derived from nociception including acute pain, tissue injury pain and nerve injury pain. Adenosine modulates the pain response by stimulating adenosine A₁ receptors present in the dorsal root of the spinal cord and higher brain centers (supraspinal mechanisms). Adenosine A₁ agonists have been shown to be an effective treatment for pain in animal pain models. However, A₁ agonists also cause cardiovascular side effects and CNS side effects such as heart block, hypotension and sedation.

The activation of adenosine A₁ receptors by the allosteric adenosine A₁ receptor enhancer, (2-amino-4,5,6,7-tetrahydrobenzo[b]thiophen-3-yl)(4-chlorophenyl)methanone has been demonstrated to reduce inflammatory and neuropathic pain and shown to be orally effective and devoid of the adverse side effects associated with administration of adenosine as disclosed in U.S. Pat. No. 6,248,774 and No. 6,489,356, the contents of which are herein incorporated by reference in their entirety.

Allosteric adenosine A₁ receptor enhancers are a class of compounds that appear to enhance adenosine A₁ receptor function by stabilizing the high affinity state of the receptor-G-protein complex. This property may be measured as an increase in radioligand binding to the adenosine A₁ receptor. An enhancer that increases agonist binding can do so by either accelerating the association of an agonist to the receptor, or by retarding the dissociation of the “receptor-ligand” complex and, therefore, must bind to a site different from the agonist recognition site. This putative site is termed the allosteric site, and presumably, compounds that bind to this site and enhance the agonist effect are termed as “allosteric enhancers”.

As indicated herein above, (2-amino-4,5,6,7-tetrahydrobenzo[b]thiophen-3-yl)(4-chlorophenyl)methanone is a known compound, and may be prepared from readily available starting materials using methods disclosed in U.S. Pat. No. 6,323,214 and No. 6,727,258, or as specifically described by Corral et al., Afinidad 1978, 35(354), 129-33.

### SUMMARY OF THE INVENTION

The present invention provides crystal forms of (2-amino-4,5,6,7-tetrahydrobenzo[b]thiophen-3-yl)(4-chlorophenyl)methanone of the formula

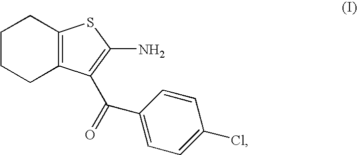

also known as T-62; processes for the production of such crystal forms; pharmaceutical compositions comprising such crystal forms; and methods of treating diseases or conditions modulated by the adenosine A₁ receptor, in particular neuropathic pain, in a mammal in need thereof, by employing such crystal forms, or pharmaceutical compositions comprising such.

It is in general desirable that a pharmaceutical product containing a crystalline drug substance has a composition which is well defined and stable in terms of the crystal form of the active ingredient. Conversion of one crystalline form into unknown amounts of different crystalline or amorphous forms during processing or storage is undesirable, and in many cases would be regarded as analogous to the appearance of unquantified amounts of impurities in the product. Therefore, it is generally desirable to manufacture the drug substance in the most stable solid state form, thereby minimizing the possibility of less stable forms being generated during storage. However, these different solid state forms (polymorphs) may offer advantages over the most stable form, such as enhanced solubility, more rapid dissolution, improved bioavailability, improved handling during manufacturing, and reduced hygroscopicity, any of which may make them more desirable than the most stable solid state form. These differences in physicochemical properties among the polymorphs of a drug substance are well known to those skilled in the art, and have been discussed widely in the literature, e.g., in “Polymorphism in Pharmaceutical Solids”, edited by Harry G. Brittain. Vol. 95, Drugs and the Pharmaceutical Sciences, Marcel Dekker, Inc. 270 Madison Avenue, New York, N.Y. 10016 (1999).

Other objects features, advantages and aspects of the present invention will become apparent to those skilled in the art from the following description, appended claims and accompanying drawings. It should be understood, however, that the following description, appended claims, drawings and specific examples, while indicating preferred embodiments of the present invention, are given by way of illustration only. Various changes and modifications within the spirit and scope of the disclosed invention will become readily apparent to those skilled in the art from reading the following.

### BRIEF DESCRIPTION OF THE DRAWINGS

FIG. 1 shows thermal analysis of Form I crystals of (2-amino-4,5,6,7-tetrahydrobenzo[b]thiophen-3-yl)(4-chlorophenyl)methanone: top line is for thermogravimetric data and bottom line is for differential scanning calorimetry data;

FIG. 2 shows a powder X-ray diffraction diagram of Form I crystals of (2-amino-4,5,6,7-tetrahydrobenzo[b]thiophen-3-yl)(4-chlorophenyl)methanone;

FIG. 3 shows an infrared absorption spectrum of Form I crystals of (2-amino-4,5,6,7-tetrahydrobenzo[b]thiophen-3-yl)(4-chlorophenyl)methanone;

FIG. 4 shows a Raman spectrum of Form I crystals of (2-amino-4,5,6,7-tetrahydrobenzo[b]thiophen-3-yl)(4-chlorophenyl)methanone;

FIG. 5. shows thermal analysis of Form II crystals of (2-amino-4,5,6,7-tetrahydrobenzo[b]thiophen-3-yl)(4-chlorophenyl)methanone: top line is for thermogravimetric data and bottom line is for differential scanning calorimetry data;

FIG. 6 shows a powder X-ray diffraction diagram of Form II crystals of (2-amino-4,5,6,7-tetrahydrobenzo[b]thiophen-3-yl)(4-chlorophenyl)methanone;

FIG. 7 shows an infrared absorption spectrum of Form II crystals of (2-amino-4,5,6,7-tetrahydrobenzo[b]thiophen-3-yl)(4-chlorophenyl)methanone;

FIG. 8 shows a Raman spectrum of Form II crystals of (2-amino-4,5,6,7-tetrahydrobenzo[b]thiophen-3-yl)(4-chlorophenyl)methanone;

FIG. 9 shows thermal analysis of Form III crystals of (2-amino-4,5,6,7-tetrahydrobenzo[b]thiophen-3-yl)(4-chlorophenyl)methanone: top line is for thermogravimetric data, bottom line is for differential scanning calorimetry data using crimped pan and dashed line for using hermetically sealed pan;

FIG. 10 shows a powder X-ray diffraction diagram of Form III crystals of (2-amino-4,5,6,7-tetrahydrobenzo[b]thiophen-3-yl)(4-chlorophenyl)methanone;

FIG. 11 shows an infrared absorption spectrum of Form III crystals of (2-amino-4,5,6,7-tetrahydrobenzo[b]thiophen-3-yl)(4-chlorophenyl)methanone;

FIG. 12 shows a Raman spectrum of Form III crystals of (2-amino-4,5,6,7-tetrahydrobenzo[b]thiophen-3-yl)(4-chlorophenyl)methanone;

FIG. 13 shows thermal analysis of Form IV crystals of (2-amino-4,5,6,7-tetrahydrobenzo[b]thiophen-3-yl)(4-chlorophenyl)methanone: top line is for thermogravimetric data and bottom line is for differential scanning calorimetry data; and

FIG. 14 shows a powder X-ray diffraction diagram of Form IV crystals of (2-amino-4,5,6,7-tetrahydrobenzo[b]thiophen-3-yl)(4-chlorophenyl)methanone.

### DETAILED DESCRIPTION OF THE INVENTION

As described herein above, the present invention provides crystal forms of the allosteric adenosine A₁ receptor enhancer, (2-amino-4,5,6,7-tetrahydrobenzo[b]thiophen-3-yl)(4-chlorophenyl)methanone of the formula

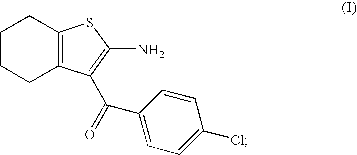

also known as T-62; processes for the production of such crystal forms; pharmaceutical compositions comprising such crystal forms; and methods of treating diseases or conditions modulated by the adenosine A₁ receptor, in particular neuropathic pain, by employing such crystal forms, or pharmaceutical compositions comprising such.

As employed throughout the description and appended claims, the term “crystals” or “crystal forms” of the instant invention refers to, as appropriate, crystal forms of (2-amino-4,5,6,7-tetrahydrobenzo[b]thiophen-3-yl)(4-chlorophenyl)methanone, i.e., T-62, designated as Form I, Form II, Form III and Form IV as defined herein below, and are substantially free of all other alternative crystalline and amorphous forms.

The term “substantially free” when referring to a designated crystal form of T-62 means that the designated crystal form contains less than 20% (by weight) of any alternate polymorphic form(s) of T-62, preferably less than 10% (by weight) of any alternate polymorphic form(s) of T-62, more preferably less than 5% (by weight) of any alternate polymorphic form(s) of T-62, and most preferably less than 1% (by weight) of any alternate polymorphic forms of T-62.

The crystal forms of the present invention may be characterized by measuring at least one of the following physico-chemical properties: 1) a melting point; 2) a X-ray powder diffraction pattern; 3) an infrared absorption spectrum; and/or 4) a Raman spectrum.

The melting points (m.p.) may be measured by Differential Scanning Calorimetry (DSC) method using a TA Instruments differential scanning calorimeter 2920, or a similar instrument. The sample is placed into an aluminium DSC pan, and the weight is recorded. The pan is covered with a lid and then crimped or hermetically sealed. Each sample is heated under nitrogen purge at a rate of 10° C./min. Indium metal is used as the calibration standard. Reported temperatures are at the transition onset.

X-ray powder diffraction (XRPD) analyses may be performed, e.g., using a Shimadzu XRD-6000 X-ray powder diffractometer or an Inel XRG-3000 diffractormeter equipped with a CSP (Curved Position Sensitive) detector with a 20 range of 120°.

For the Shimadzu XRD-6000 X-ray powder diffractometer, or a similar instrument, Cu Kα radiation is used. The instrument is equipped with a fine focus X-ray tube. The tube voltage and amperage are set to 40 kV and 40 mA, respectively. The divergence and scattering slits are set at 10 and the receiving slit is set at 0.15 mm. Diffracted radiation is detected by a NaI scintillation detector. A theta-two theta continuous scan at 3°/min (0.4 sec/0.02° step) from 2.5 to 40 °2θ is used. A silicon standard is analyzed to check the instrument alignment. Data are collected and analyzed using XRD-6000 v. 4.1. Samples are prepared for analysis by placing them in an aluminum holder with a flat silicon insert.

For the Inel XRG-3000, or a similar instrument, real time data are collected using Cu-Kα radiation starting at approximately 4 °2θ, at a resolution of 0.03 °2θ. The tube voltage and amperage are set to 40 kV and 30 mA, respectively. The pattern is displayed from 2.5-40 °2θ. Samples are prepared for analysis by packing them into thin-walled glass capillaries. The capillaries are sometimes sealed by heat, or with Critoseal. Each capillary is mounted onto a goniometer head that is motorized to permit spinning of the capillary during data acquisition. The samples are generally analyzed for 5 min. Analysis time is 20-30 min for pattern indexing, which requires higher resolution. Instrument calibration is performed using a silicon reference standard.

Infrared absorption spectra may be acquired on a Magna 860® Fourier transform infrared (FT-IR) spectrophotometer (Thermo Nicolet) equipped with an Ever-Glo mid/far IR source, a potassium bromide (KBr) beamsplitter, and a deuterated triglycine sulfate (DTGS) detector, or a similar instrument. A diffuse reflectance accessory (the Collector™, Thermo Spectra-Tech) may be used for sampling. Each spectrum represents 128 co-added scans, collected at a spectral resolution of 4 cm⁻¹. Sample preparation consists of placing the sample into a 3-mm or a 13-mm diameter cup and leveling the material with a glass slide. A background data set is acquired with an alignment mirror in place. A Log 1/R(R reflectance) spectrum is acquired by taking a ratio of these two data sets against each other. Wavelength calibration is performed using polystyrene.

FT-Raman spectra may be acquired on a FT-Raman 960 spectrometer (Thermo Nicolet), or a similar instrument. This spectrometer uses an excitation wavelength of 1064 nm. Approximately 0.5 W of Nd:YVO₄ laser power is used to irradiate the sample. The Raman spectra are measured with an indium gallium arsenide (InGaAs) detector. The samples are prepared for analysis by placing the material in a glass tube (sometimes a thin-walled capillary) and positioning the tube in a gold-coated holder in the accessory. A total of 128 sample scans are collected at a spectral resolution of 4 cm⁻¹ using Happ-Genzel apodization. Wavelength calibration is performed using sulfur and cyclohexane.

One of ordinary skill in the art will appreciate that the physico-chemical properties discussed herein above may be obtained with a measurement error that is dependent upon the measurement conditions employed. In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, e.g., ±0.2 °2θ, and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray diffraction patterns depicted in the accompanying Figures disclosed herein. Any crystal forms that provide X-ray diffraction patterns substantially identical to those disclosed in the accompanying Figures fall within the scope of the present invention. The ability to ascertain substantial identities of X-ray diffraction patterns is within the purview of one of ordinary skill in the art. A discussion of the theory of powder X-ray diffraction patterns can be found, e.g., in “X-Ray Diffraction Procedures” by Klug and Alexander, J. Wiley, New York (1974).

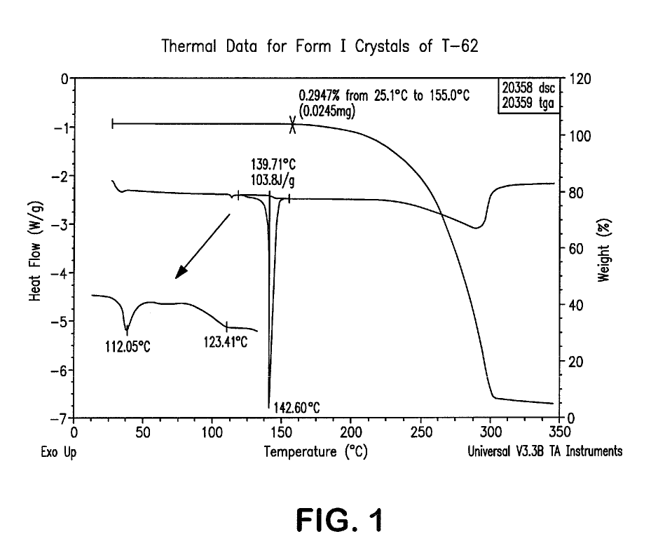

In one aspect, the present invention provides a crystal form of T-62, designated herein as Form I, having a melting point of about 143° C. as measured by the DSC method described herein above and depicted in FIG. 1.

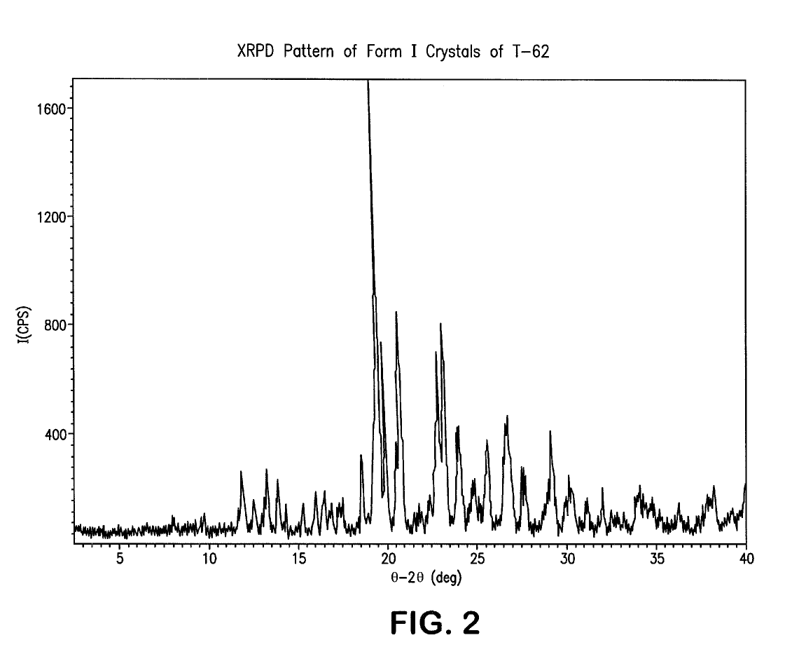

An example of an X-ray diffraction pattern exhibited by a Form I crystal form is substantially identical to that depicted in FIG. 2, having characteristic peaks, expressed in degrees 2-theta (2θ), of about 19.6±0.2, 19.9±0.2, 20.8±0.2, 22.9±0.2 and 23.3±0.2.

The present invention also provides a Form I crystal form that exhibits a X-ray diffraction pattern substantially the same as that depicted in FIG. 2, having characteristic diffraction peaks expressed in degrees 2-theta, and relative intensities (I/I₁) of approximately the values shown in Table 1 herein below:

TABLE 1
Form I crystals of T-62

| Angle (deg 2θ) | Relative intensity (I/I₁) |
|---|---|
| 11.9 ± 0.2 | 11 |
| 13.3 ± 0.2 | 13 |
| 13.9 ± 0.2 | 11 |
| 18.6 ± 0.2 | 14 |
| 19.6 ± 0.2 | 100 |
| 19.9 ± 0.2 | 40 |
| 20.8 ± 0.2 | 48 |
| 22.9 ± 0.2 | 37 |
| 23.3 ± 0.2 | 46 |
| 24.0 ± 0.2 | 22 |
| 25.7 ± 0.2 | 18 |
| 26.7 ± 0.2 | 20 |
| 27.6 ± 0.2 | 13 |
| 29.2 ± 0.2 | 23 |
| 38.2 ± 0.2 | 10 |

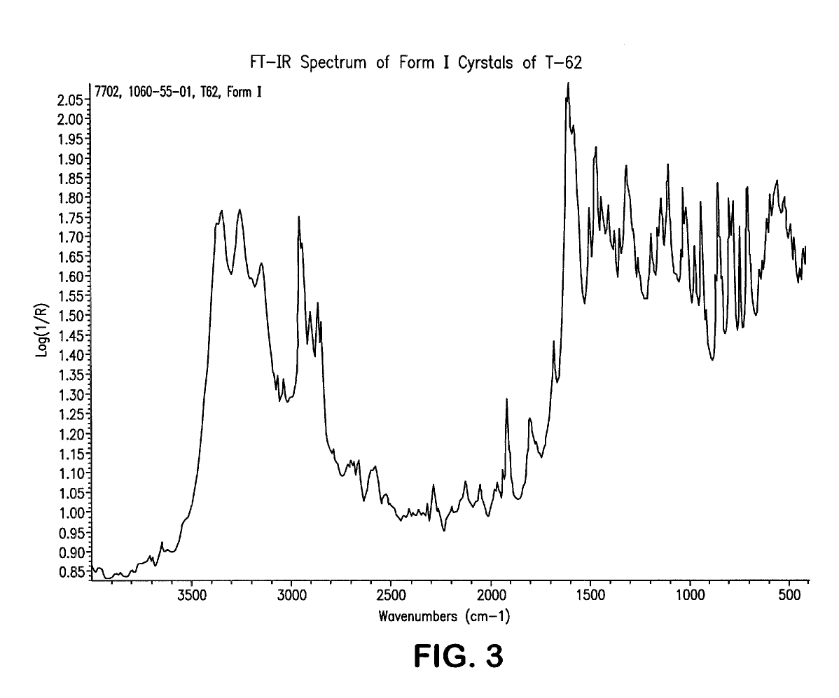

An example of an infrared absorption spectrum of a Form I crystal form obtained by the diffuse reflectance method is shown in FIG. 3, and is characterized by absorption bands at about 3330±2, 3239±2, 2943±2, 1908±2 and 927±2 cm⁻¹.

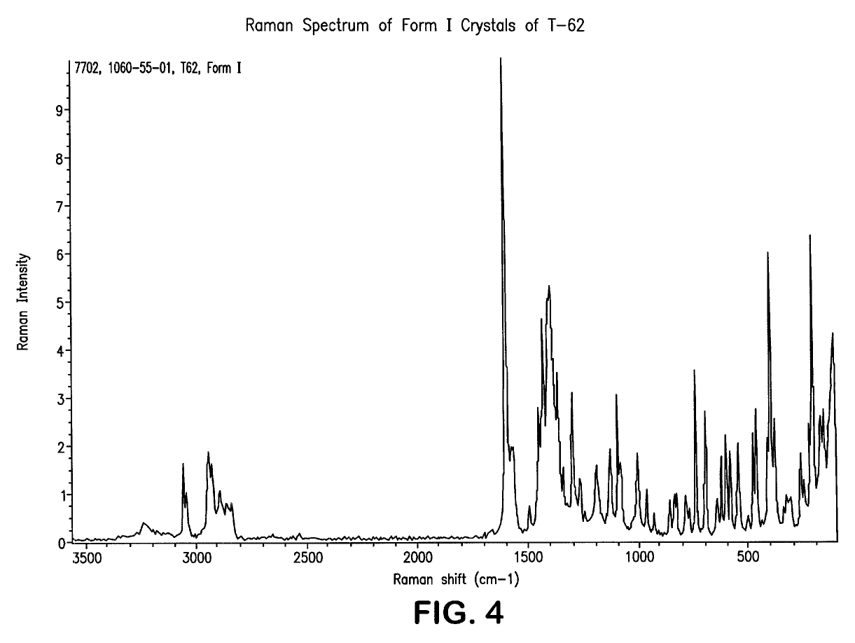

An example of a FT-Raman spectrum of a Form I crystal form obtained by the method described herein above is shown in FIG. 4, and is characterized by Raman shifts at about 3060±2, 2944±2, 1596±2, 1426±2 and 1391±2 cm⁻¹.

Form I crystal form of T-62 is anhydrous and moisture sorption/desorption analysis of Form I crystals show no significant weight gain or loss indicating Form I is non-hygroscopic. The crystal form remains the same after moisture sorption/desorption analysis.

Moisture sorption/desorption analyses may be performed on a VTI SGA-100 Vapor Sorption Analyzer, or a similar instrument. Sorption and desorption data are collected, e.g., over a range of 5 to 95% relative humidity (RH) at 10% RH intervals under a nitrogen purge. Samples need not to be dried prior to analysis. Equilibrium criteria used for analysis are, e.g., less than 0.01% weight change in 5 min, with a maximum equilibration time of 3 h if the weight criterion is not met. NaCl and PVP may be used as calibration standards.

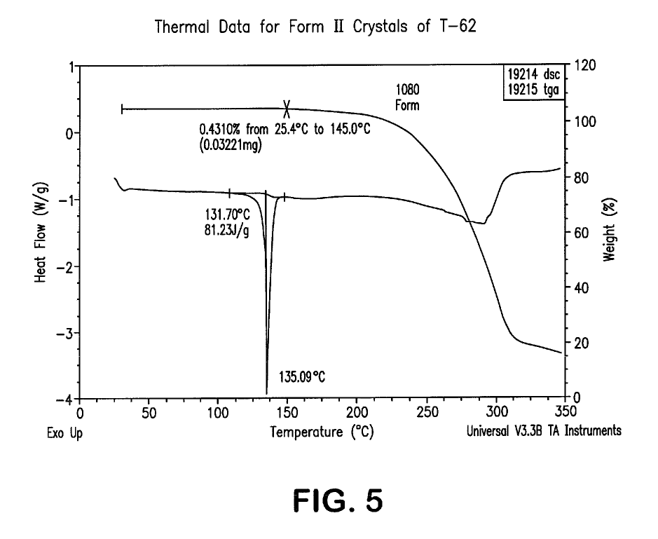

In another aspect, the present invention provides a crystal form of T-62, designated herein as Form II, having a melting point of about 135° C. as measured by the DSC method described herein above and depicted in FIG. 5.

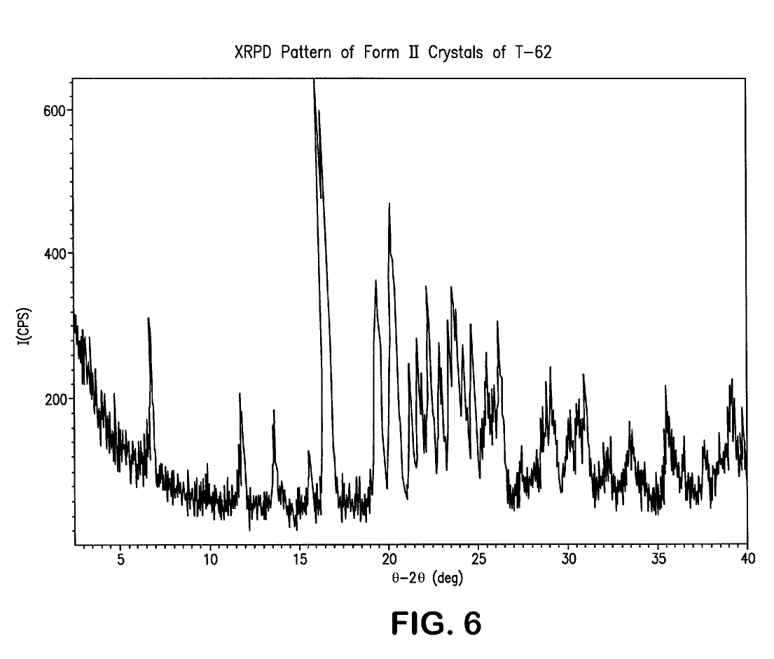

An example of an X-ray diffraction pattern exhibited by a Form II crystal form is substantially identical to that depicted in FIG. 6, having characteristic peaks, expressed in degrees 2-theta (20), of about 16.5±0.2, 19.4±0.2, 20.3±0.2, 22.2±0.2, 23.6±0.2 and 26.2±0.2. The present invention also provides a Form II crystal form that exhibits a X-ray diffraction pattern substantially the same as that depicted in FIG. 6, having characteristic diffraction peaks expressed in degrees 2-theta, and relative intensities (I/I₁) of approximately the values shown in Table 2 herein below:

TABLE 2
Form II crystals of T-62

| Angle (deg 2θ) | Relative intensity (I/I₁) |
|---|---|
| 6.7 ± 0.2 | 26 |
| 11.6 ± 0.2 | 24 |
| 13.5 ± 0.2 | 17 |
| 15.5 ± 0.2 | 10 |
| 16.5 ± 0.2 | 100 |
| 19.4 ± 0.2 | 52 |
| 20.3 ± 0.2 | 68 |
| 21.1 ± 0.2 | 24 |
| 21.6 ± 0.2 | 28 |
| 22.2 ± 0.2 | 37 |
| 22.8 ± 0.2 | 24 |
| 23.6 ± 0.2 | 40 |
| 24.6 ± 0.2 | 27 |
| 26.2 ± 0.2 | 34 |
| 28.5 ± 0.2 | 12 |
| 29.0 ± 0.2 | 18 |
| 30.0 ± 0.2 | 13 |
| 30.9 ± 0.2 | 24 |
| 33.4 ± 0.2 | 14 |
| 35.4 ± 0.2 | 20 |
| 37.5 ± 0.2 | 10 |
| 39.0 ± 0.2 | 19 |

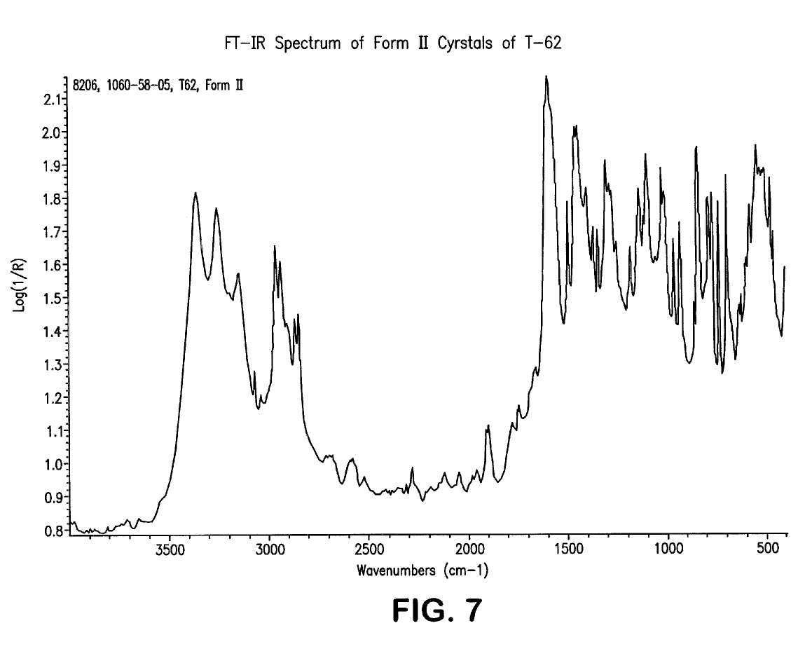

An example of an infrared absorption spectrum of a Form II crystal form obtained by the diffuse reflectance method is shown in FIG. 7, and is characterized by absorption bands at about 3344±2, 3243±2, 2955±2, 2930±2, 1895±±2 and 1172±2 cm⁻¹.

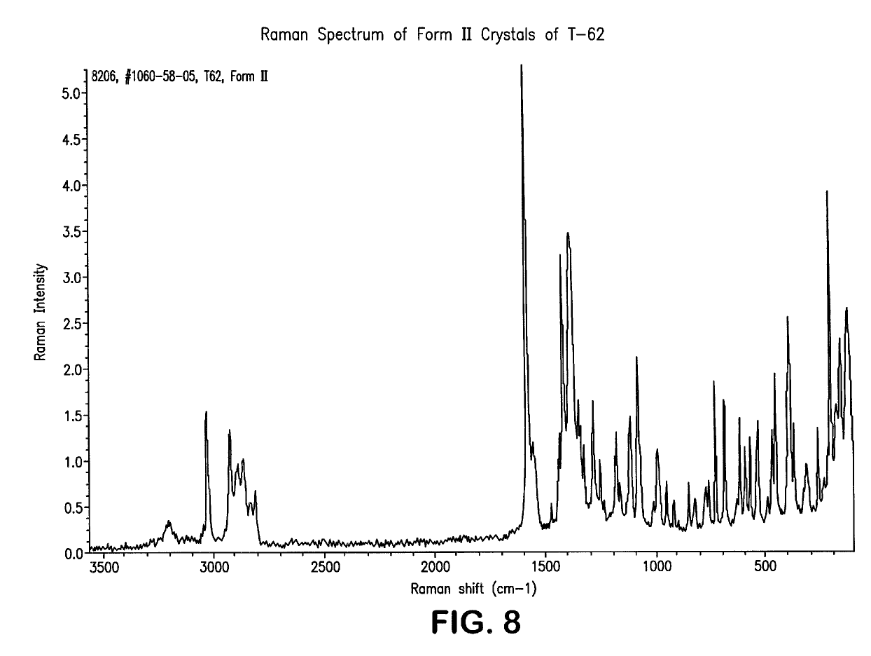

An example of a FT-Raman spectrum of a Form II crystal form obtained by the method described herein above is shown in FIG. 8, and is characterized by Raman shifts at about 3065±2, 2957±2, 1595±2, 1430±2 and 1394±2 cm⁻¹.

Form II crystal form of T-62 is anhydrous and moisture sorption/desorption analysis of Form II crystals shows negligible weight gain (0.1% gain at 95% RH) indicating Form II is non-hygroscopic. The crystal form remains the same after moisture sorption/desorption analysis.

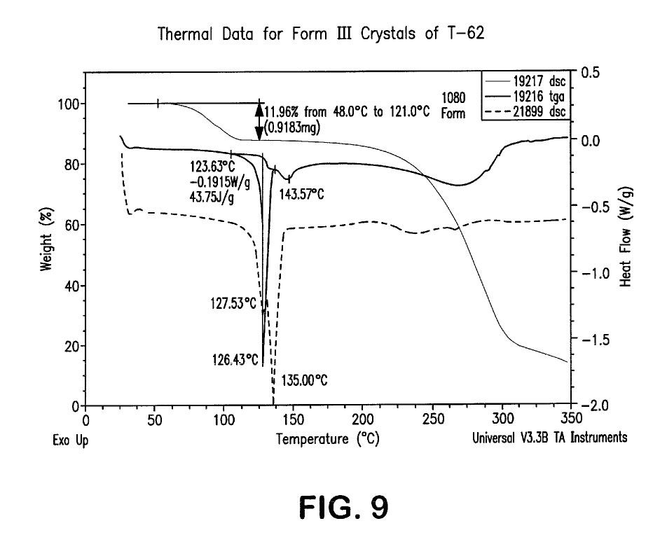

In yet another aspect, the present invention provides a crystal form of T-62, designated herein as Form III, having a melting point in the range of about 126-135° C. as measured by the DSC method described herein above and depicted in FIG. 9.

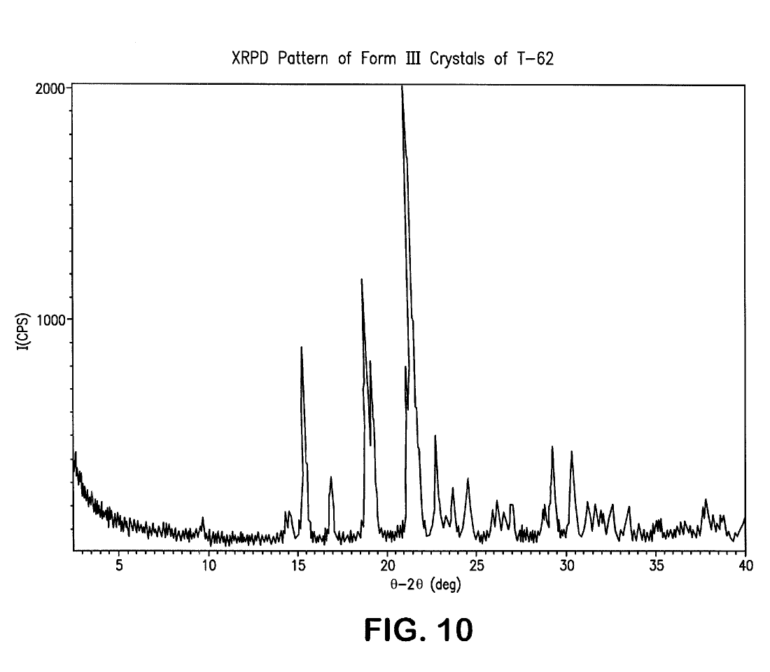

An example of an X-ray diffraction pattern exhibited by a Form III crystal form is substantially identical to that depicted in FIG. 10, having characteristic peaks, expressed in degrees 2-theta (2θ), of about 15.5±0.2, 19.1±0.2, 19.4±0.2, 21.3±0.2 and 21.7±0.2.

The present invention also provides a Form III crystal form that exhibits a X-ray diffraction pattern substantially the same as that depicted in FIG. 10, having characteristic diffraction peaks expressed in degrees 2-theta, and relative intensities (I/I₁) of approximately the values shown in Table 3 herein below:

TABLE 3
Form III crystals of T-62

| Angle (deg 2θ) | Relative intensity (I/I₁) |
|---|---|
| 15.5 ± 0.2 | 40 |
| 16.9 ± 0.2 | 12 |
| 19.1 ± 0.2 | 57 |
| 19.4 ± 0.2 | 34 |
| 21.3 ± 0.2 | 34 |
| 21.7 ± 0.2 | 100 |
| 22.9 ± 0.2 | 21 |
| 24.6 ± 0.2 | 12 |
| 29.4 ± 0.2 | 19 |
| 30.5 ± 0.2 | 18 |

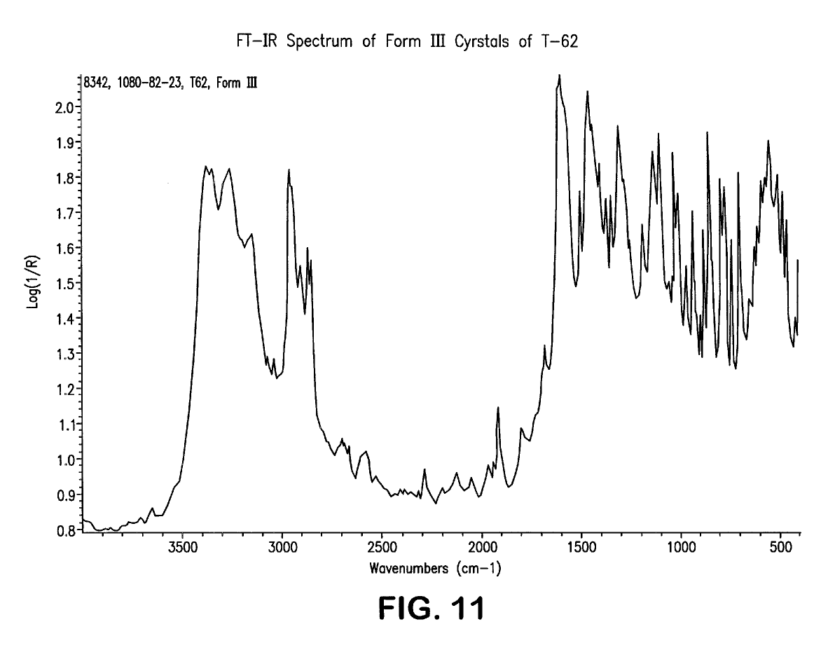

An example of an infrared absorption spectrum of a Form III crystal form obtained by the diffuse reflectance method is shown in FIG. 11, and is characterized by absorption bands at about 3247±2, 2941±2, 1908±2 and 922±2 cm⁻¹.

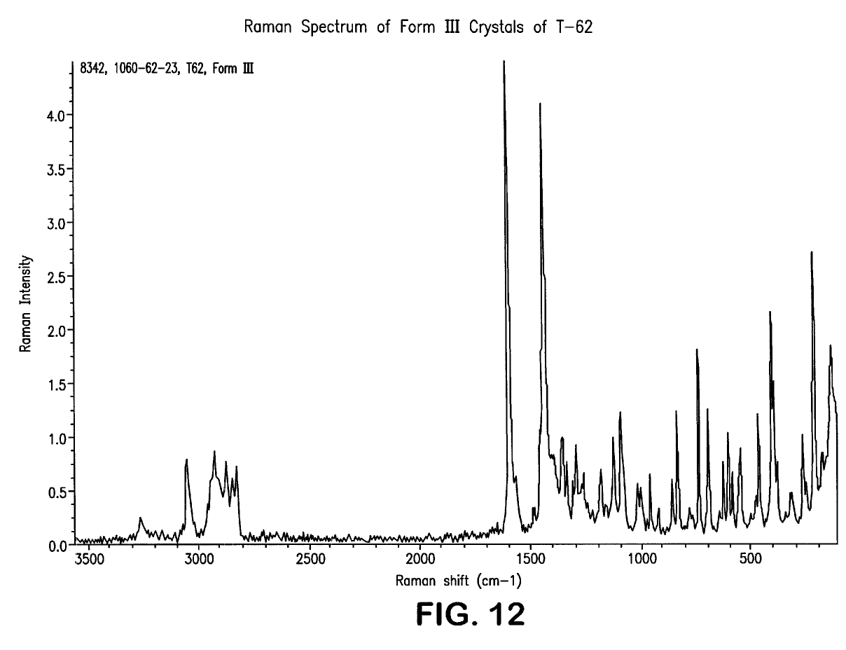

An example of a FT-Raman spectrum of a Form III crystal form obtained by the method described herein above is shown in FIG. 12, and is characterized by Raman shifts at about 3068±2, 2939±2, 1596±2 and 1436±2 cm⁻¹.

Crystal Form III is a dioxane solvate, and thermogravimeteric (TG) analyses of Form III crystals shows about 12% weight loss which corresponds to about 0.5 mole of dioxane (FIG. 9). The solvent is lost under high humidity and the crystal form converts to Form I crystal form. Form III crystal form also converts to Form I crystals after losing dioxane upon heating.

Thermogravimetric analyses (TG) may be performed using TA Instruments 2950 thermogravimetric analyzer, or a similar instrument. Each sample is placed in an aluminium sample pan and inserted into the TG furnace. Samples are heated under nitrogen at a rate of 10° C./min. Nickel and Alumel™ are used as the calibration standards.

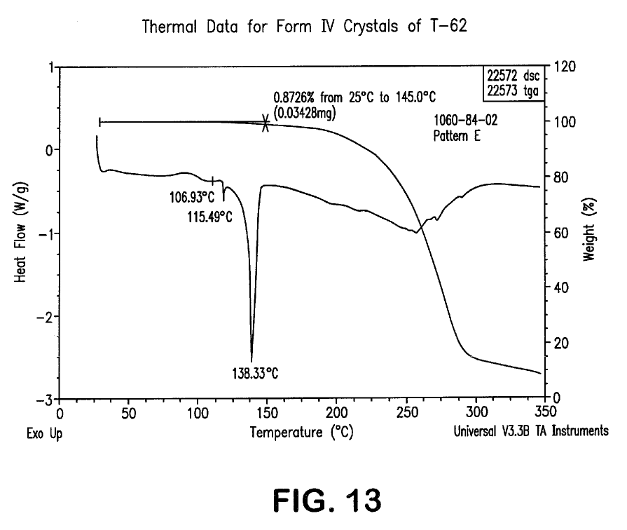

In yet another aspect, the present invention provides a crystal form of anhydrous T-62, designated herein as Form IV, having a melting point of about 138° C. as measured by the DSC method described herein above and depicted in FIG. 13.

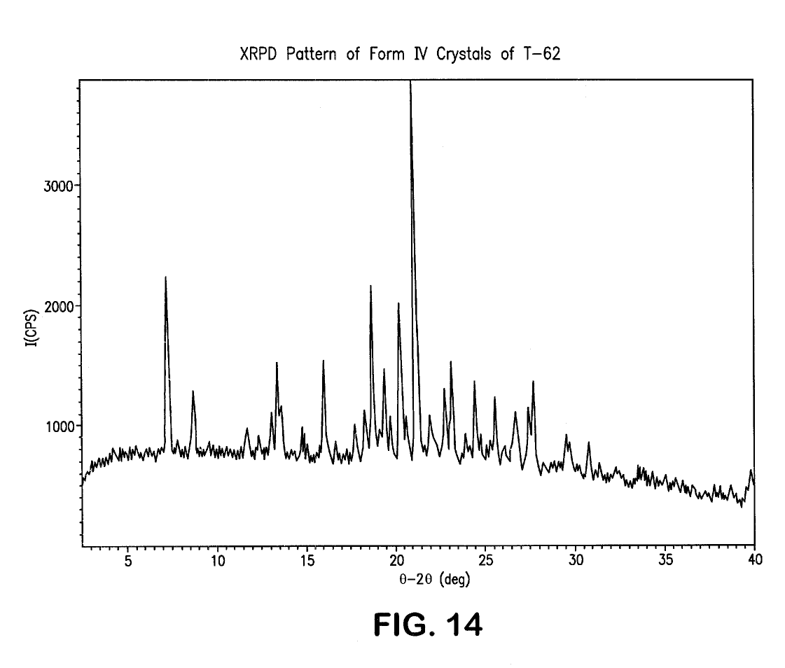

An example of an X-ray diffraction pattern exhibited by a Form IV crystal form is substantially identical to that depicted in FIG. 14, having characteristic peaks, expressed in degrees 2-theta (2θ), of about 7.4±0.2, 18.8±0.2, 20.4±0.2 and 21.3±0.2. The present invention also provides a Form IV crystal form that exhibits a X-ray diffraction pattern substantially the same as that depicted in FIG. 14, having characteristic diffraction peaks expressed in degrees 2-theta, and relative intensities (I/I₁) of approximately the values shown in Table 4 herein below:

TABLE 4
Form IV crystals of T-62

| Angle (deg 2θ) | Relative intensity (I/I₁) |
|---|---|
| 7.4 ± 0.2 | 45 |
| 8.8 ± 0.2 | 15 |
| 13.2 ± 0.2 | 10 |
| 13.6 ± 0.2 | 21 |
| 16.1 ± 0.2 | 25 |
| 18.4 ± 0.2 | 11 |
| 18.8 ± 0.2 | 43 |
| 19.5 ± 0.2 | 21 |
| 20.4 ± 0.2 | 38 |
| 21.3 ± 0.2 | 100 |
| 22.1 ± 0.2 | 10 |
| 23.2 ± 0.2 | 25 |
| 24.5 ± 0.2 | 20 |
| 25.6 ± 0.2 | 15 |
| 26.8 ± 0.2 | 13 |
| 27.8 ± 0.2 | 24 |

Likewise, Form IV crystal form of T-62 converts to Form I crystals upon heating.

The crystal forms of T-62 are preferably substantially stable to grinding. Stability to grinding may be assessed by measurement of an appropriate physical property before and after grinding. Where the physical property remains substantially unchanged, substantial stability to grinding is indicated. Suitable physico-chemical properties for measurement include a melting point, a X-ray diffraction pattern, an infra red absorption spectrum and a Raman spectrum, particularly the X-ray diffraction pattern. For example, Form I crystal form of T-62 is stable to mechanical milling and cryogrinding as indicated by XRPD analyses of the milled and ground material, respectively.

Stability to mechanical grinding/milling may be tested, e.g., by employing a small stainless steel cylinder. The cylinder is charged with a weighted amount of a crystal form of T-62 and a stainless steel ball is added, the cylinder is capped and the unit is placed on a Retsch Mixer Mill (Type MM 200) for a total of 3 min at a frequency of 30/s (ball-milling).

Stability to cryogrinding may be tested, e.g., by employing a polycarbonate freezer mill tube. The tube is charged with a weighted amount of a crystal form of T-62 and a stainless steel rod is added, the tube is capped and the unit is shaken in a SPEX Certiprep 6750 Freezer Mill for a total of 0.6 min in liquid nitrogen at a rate of 10 Hz.

In a further aspect, the present invention provides methods for the production of different crystal forms of T-62 wherein the method comprises dissolving T-62, in any of its forms, including amorphous forms, in a suitable solvent, including mixed solvents, forming the T-62 crystals, isolating and drying the crystal form of T-62, including solvates, e.g., a dioxane hemi-solvate of T-62.

In one embodiment of the present invention, crystal forms of T-62 may be produced by dissolving of T-62, in any its forms, in a solvent to form a solution in which solvent T-62 is suitably soluble at an elevated temperature ranging from about 30° C. to the boiling point of the solvent, and in which solvent T-62 is only poorly soluble at a lower temperature ranging from about −78° C. to about 30° C., cooling the solution to the lower temperature to induce precipitation of a crystal form of T-62, isolating and drying the precipitated crystal form of T-62, in particular the Form I crystal form of T-62.

Solvents in which T-62 is suitably soluble at an elevated temperature ranging from about 30° C. to the boiling point of the solvent, and in which T-62 is only poorly soluble at a lower temperature ranging from about −78° C. to about 30° C. include, but are not limited to, acetonitrile (ACN), dioxane, tetrahydrofuran (THF), esters such as methyl and ethyl acetate (EtOAc), ketones such as acetone and methylethylketone (MEK), lower alcohols such as methanol (MeOH) and ethanol (EtOH), and the like. Acetonitrile is particularly effective. Preferred mixed solvents include a mixture of a polar solvent such as a lower alcohol, e.g., methanol and ethanol, preferably ethanol, with water. When a mixed solvent is employed, preferably a mixture of methanol or ethanol with water, more preferably a mixture of ethanol with water, the concentration of the polar solvent in the solvent mixture is generally about 50% by volume or more. Preferably, the concentration of the polar solvent in the solvent mixture is between 50% and 95% by volume, more preferably between 85% and 95% by volume, and most preferably about 95% by volume. The dissolution temperature preferably ranges from about 60° C. to about 80° C., and more preferably the dissolution temperature ranges is about 78° C. The amount of T-62 in the solvent ranges preferably from about 1% to about 50% by weight of the resulting mixture, more preferably from about 20% to about 30% by weight. If the amount of T-62 is more than 50% by weight then the slurry properties of the initial suspension are poor and it will be difficult to agitate the mixture and dissolve the solid. On the other hand, it is not efficient in terms of the volume of the solvent required to use less than 1% of T-62 by weight. The lower temperature to which the solution of T-62 is cooled to induce precipitation of the desired crystal form of T-62 ranges from about −78° C. to about 30° C., preferably from about 4° C. to about 30° C. It should be noted that the rate in which the solution is cooled to the lower temperature may also affect which crystal form of T-62 is produced, e.g., when a solution of T-62 in acetonitrile at 60° C. is allowed to cool slowly (SC), e.g., to room temperature, Form II crystals of T-62 are obtained, whereas cooling the solution quickly, i.e., crash cooling (CC), e.g., by placing it directly in an ice-water or dry ice/acetone bath produces Form I crystals. Furthermore, it may be advantageous to add seed crystals of the desired form to the solution to further aid precipitation. The resulting mixture may then be maintained at the lower temperature for a time sufficient to assure complete precipitation of the desired form of T-62 crystals.

In a specific embodiment, the solvent in which T-62 is suitably soluble is a 95:5 mixture of ethanol and water, i.e., 95% of ethanol by volume. A solution of approximately 250 mg/mL of T-62, in any of its forms, is heated at about 78° C. with stirring to achieve a complete dissolution. The clear solution is then cooled to a temperature of about 4° C. to induce precipitation. The precipitated crystals are isolated by vacuum filtration and dried to give Form I crystals of T-62.

Alternatively, crystal forms of T-62 can be produced by a method wherein the method comprises dissolving T-62, in any of its forms, in a solvent, referred to herein as a first solvent (S), to form a solution, in which solvent T-62 is readily soluble at a temperature ranging from room temperature to the boiling point of the solvent, preferably 75° C., treating the solution with another solvent, referred to herein as a second solvent (an anti-solvent, AS), which is miscible with the first solvent and in which T-62 is only poorly soluble, to induce precipitation of a crystal form of T-62, isolating and drying the precipitated crystal form of T-62, in particular the Form I crystal form of T-62, including solvates such as a dioxane hemi-solvate.

First solvents in which T-62 is readily soluble, i.e., in amounts of at least 5% by weight at the dissolution temperature, include lower alcohols, such as methanol, ethanol and isopropanol (IPA). Polar solvents such as acetone, tetrahydrofuran (THF), dioxane and methylethylketone (MEK) can also be effective when used as the first solvent. Second solvents in which T-62 is only poorly soluble, i.e., in amounts of 2% by weight or less, include but are not limited to, water and hexane. The dissolution temperature, i.e., the temperature in which T-62 is dissolved in the first solvent, ranges preferably from room temperature to about the boiling point of the solvent, more preferably from room temperature to about 75° C., and most preferably the dissolution temperature is 75° C. The amount of T-62 dissolved in the first solvent ranges preferably from 5 to 50% by weight of the resulting solution. The solution of T-62 in the first solvent may be added to the second solvent, or the second solvent may be added to the solution of T-62 in the first solvent and, if desired, the resulting mixture may be cooled to further induce precipitation. It may be advantageous to filter the solution of T-62 in the first solvent prior to induction of precipitation. The ratio of the first solvent to the second solvent in the resulting mixture ranges preferably from about 1 to 1 to about 1 to 9 by volume. It may also be advantageous to include seed crystals in the mixture to aid precipitation of the desired T-62 crystals. Preferably, the seed crystals are included in the second solvent prior to the combination of the second solvent with the solution of T-62 in the first solvent. The second solvent may be cooled to a lower temperature ranging from about −78° C. to room temperature prior to mixing, preferably the lower temperature ranges from about −78° C. to about 20° C. The resulting mixture containing the desired crystal form of T-62 may then be maintained at the lower temperature, or at a temperature ranging from about −20° C. to about 20° C., for a time sufficient to assure complete precipitation of the desired T-62 crystals.

In a specific embodiment, the first solvent is dioxane and the second solvent is hexane. T-62, in any of its forms, is dissolved in the first solvent, e.g., at room temperature while stirring to form a clear solution, followed by addition of the resulting solution to the chilled second solvent. The precipitated crystals may then be isolated by vacuum filtration to give Form III crystals of T-62.

In another specific embodiment, the first solvent is IPA and the second solvent is water. T-62, in any of its forms, is dissolved in the first solvent, e.g., at a temperature of about 75° C. while stirring to form a clear solution, followed by addition of the second solvent to the solution of T-62 in the first solvent. The mixture is then cooled to about 20° C. to further induce precipitation, and finally to about 10° C. to assure complete precipitation of the T-62 crystals. The precipitated crystals may then be isolated by vacuum filtration to give Form I crystals of T-62.

Alternatively to cooling and/or addition of anti-solvent to induce precipitation, a solution of T-62, e.g., any of those described herein above, may be concentrated by evaporation to certain fraction of the original volume to induce precipitation of the desired crystal form of T-62. Preferably, the solution of T-62 is evaporated to dryness at an ambient temperature. It should be noted that the rate in which the solution is concentrated may also affect which crystal form of T-62 is produced, i.e., fast evaporation (FE) may be carried out without covering the vessel containing the solution of T-62, or said vessel may be covered with a foil perforated with pinholes to affect slow evaporation (SE).

In a specific embodiment, T-62, in any of its forms, is dissolved in ethyl acetate at room temperature to form a clear solution. The resulting solution of T-62 is then allowed to evaporate to dryness at room temperature without cover (fast evaporation) to produce Form II crystals of T-62.

Conventional methods, such as heating, sonication and stirring, may be used to aid dissolution of T-62. T-62, in any of its forms, including solvates such as a dioxane hemi-solvate, may be added to the solvent or the solvent may be added onto T-62, stirred, and heated to a temperature ranging from room temperature to about the boiling point of the solvent to form a solution. Stirring, cooling and addition of seed crystals may be used to further induce precipitation of the desired crystal form of T-62. The precipitated crystals may be isolated by conventional methods, such as vacuum filtration or centrifugation. The crystals may be washed, preferably with a solvent or a solvent mixture consisting of solvents used in the crystallization. During isolation and washing, cooling may be applied, if so desired, preferably cooling the crystals to a temperature ranging from about −20° C. to about 8° C. The isolated T-62 crystals may be dried under atmospheric or reduced pressure, preferably under reduced pressure ranging from about 20 to about 0.1 mmHg, at an ambient temperature.

A polymorph screen may be carried out to obtain as many crystals forms as possible using a variety of solvents and techniques as described herein above. The solid samples obtained from the screen are initially observed (visually and under optical microscopy), then analyzed by XRPD. The XRPD data are then compared to each other to determine how many crystal forms are present. Once similar XRPD patterns are grouped together, each group is assigned a Form number (e.g., I, II, III and IV). One XRPD pattern from each group is designated as a “standard” pattern for comparison purposes. Many of the samples exhibit preferred orientation (PO) and selected samples are reanalyzed by Inel diffractometer. The samples that exhibit similar PO are grouped, and only a few of them are reanalyzed by Inel diffractometer to avoid redundancy. Some of the XRPD patterns may exhibit peak shifts. This peak shift on the x-axis occurs when the sample height is different than the average sample height. Higher or lower sample heights due to packing differences will cause a systematic peak shift on the x-axis. The crystal habits of the samples may be observed under optical microscopy. Some solids exhibit indescribable morphology and are denoted as “unknown”. All of the polymorph screen samples of Form I are yellow, whereas Form II samples are reddish in color and amorphous samples are purple red. The color of amorphous samples are probably due to decomposition products. A total of four crystalline forms and an amorphous form have been identified. Attempts to obtain hydrates yield only Form I crystal form. The four crystalline forms are unique and easily identified by, e.g., XRPD.

Optical microscopy may be performed using a Leica DM LP microscope equipped with a Sony DXC-970MD 3CCD camera, or a similar instrument. Samples are observed at object magnification of 20× or 40× using cross polarizers. Samples are placed on a glass slide. Images are acquired at ambient temperature using Linksys for Windows software (v.2.27). If desired, micron bars may be inserted onto the images as a reference for particle size.

Illustrative examples of preferred embodiments as obtained in a polymorph screen are summarized in Table 5 below:

TABLE 5

| Solvent | Conditions | XRPD Result | Habit |
|---|---|---|---|
| acetone | S/AS (hexane) | Form I | needles |
| ACN | CC | Form I | unknown |
| ACN | SC | Form II | unknown |
| di-n-buthyl ether | FE | Form I | dendrite |
| chloroform (CHCl₃) | FE | Form I | unknown |
| dichloromethane | S/AS (hexane) | Form I | rosette of blades |
| dioxane | S/AS (hexane) | Form III | unknown |
| EtOH | FE | Form I | plates |
| EtOH | SE | Form I | unknown |
| EtOH:H₂O - 95:5 | FE | Form I | plates |
| EtOH:H₂O - 95:5 | SC | Form I | unknown |
| EtOH:H₂O - 95:5 | CC | Form I | tabular |
| EtOH:H₂O - 85:15 | SE | Form I | plates |
| EtOAc | FE | Form II | fine fibers |
| diisopropyl ether | FE | Form I | unknown |
| IPA | FE | Form I | unknown |
| IPA | SE | Form IV | unknown |
| IPA | SC | Form I | unknown |
| IPA | S/AS (H₂O) | Form I | unknown |
| MEK | S/AS (H₂O) | Form I | unknown |
| MEK | S/AS (hexane) | Form I | rosette of plates |
| MeOH | FE | Form II | fibrous |
| MeOH | S/AS (H₂O) | Form I | unknown |
| MeOH:H₂O - 95:5 | SE | Form I | unknown |
| toluene | FE | Form I | unknown |
| THF | S/AS (H₂O) | Form I | blades |
| THF | S/AS (hexane) | Form I | unknown |
| ACN | SE | amorphous | unknown |

### Fast Evaporation (FE) and Slow Evaporation (SE):

A weight amount of T-62 (generally 50-90 mg) is treated with an appropriate amount of a test solvent to obtain complete dissolution. Dissolution is judged by visual observation. The resulting solution is filtered through a 0.2-μm nylon filter into a clean vial and left in a fume hood at ambient temperature under one of the following two conditions:

(a) fast evaporation (FE)—without cover; and

(b) slow evaporation (SE)—with a foil cover perforated with pinholes.

Some additional drying is applied to the samples that appear wet after the evaporation is completed.

### Slow Cooling (SC):

A measured amount of a test solvent is added to a weight amount of T-62. The mixture is heated and stirred on a hotplate (or in a silicon oil bath) until all of the solid has dissolved (visual observation), generally at about 60° C. The resulting solution is filtered through a 0.2-μm nylon filter into a clean vial pre-warmed to the same temperature. The heat source is turned off, and the filtered solution is allowed to cool to an ambient temperature. Samples are often allowed to stand at an ambient temperature overnight. Some of the samples are placed in a refrigerator (approximately 2° C. to 8° C.) or freezer (approximately −20° C. to −10° C.) for at least one day to encourage particle formation. Solids are harvested through vacuum filtration or decantation.

### Crash Cooling (CC):

A measured amount of T-62 is combined with a measured amount of a test solvent (or a mixture of solvents) at an elevated temperature (generally at about 60° C.) generating a clear solution (visual observation). The sample is filtered through a 0.2-μm syringe filter (pre-warmed to the same temperature). The filtered solution is then placed in an ice-water or dry ice/acetone bath. Occasionally, additional cooling is necessary, and the samples are placed in a refrigerator (approximately 2° C. to 8° C.) and/or freezer (approximately −20° C. to −10° C.). Solids are collected by vacuum filtration.

### Anti-Solvent Precipitation (S/AS):

A measured amount of T-62 is combined with a measured amount of a test solvent, generating a solution (sometimes with heating). The resulting solution is often filtered through a 0.2-μm nylon filter into a clean glass vial. A solvent in which T-62 has low solubility, an anti-solvent, is added (or the solution is added to the anti-solvent). Occasionally, the anti-solvent is chilled prior to addition. Solids are collected by vacuum filtration.

According to yet another aspect of the present invention, Form III and Form IV crystals of T-62 may be converted to the Form I crystal form of T-62 by storing the former type of T-62 crystals under atmospheric, super-atmospheric or reduced pressure at a temperature ranging from about 20° C. to about 104° C. and in relative humidity up to about 95% for a time sufficient to convert the former type crystals of T-62 crystals to the Form I crystal form of T-62.

In a specific embodiment of the present invention, Form III crystals are converted to the Form I crystal form by heating the former crystals to a temperature of about 104° C. under argon purge. In another specific embodiment of the present invention, Form IV crystals of T-62 are converted to the Form IV crystal form by heating the crystals for 2 min at 75° C. in a sealed capillary.

Accordingly, in a further aspect the present invention provides a use of the Form III and Form IV crystals for the manufacture of the Form I crystal form.

According to a still further aspect of the present invention herein, is provided a pharmaceutical composition comprising crystals of T-62, as obtainable by the methods of the present invention, in particular Form I crystals of T-62, and pharmaceutically acceptable excipients, diluents or carriers thereof.

According to a still further aspect of the present invention, herein is provided a method for manufacture of a pharmaceutical composition comprising a therapeutically effective amount of crystals of T-62, as obtainable by the methods of the present invention, in particular Form I crystals of T-62, and pharmaceutically acceptable excipients, diluents or carriers thereof.

The term “therapeutically effective amount” as used herein refers to an amount of crystals of T-62 that will elicit the desired biological or medical response of a tissue, system or a mammal (including man) that is being sought by a researcher or clinician, in particular pain management, preferably management of neuropathic pain.

The term “pain management” shall be understood herein to include the expressions “reduction of pain”, “suppression of pain”, “alleviation of pain” and “inhibition of pain” as the present invention is applicable to the alleviation of existing pain as well as the suppression or inhibition of pain which would otherwise ensue from an imminent pain-causing event.

The term “mammal, warm-blooded animal or patient” are used interchangeably herein and include, but are not limited to, humans, dogs, cats, horses, pigs, cows, monkeys, rabbits, mice and laboratory animals. The preferred mammals are humans.

In a still further aspect of the present invention, herein is provided a method for the treatment of diseases or conditions modulated by the adenosine A₁ receptor, in particular neuropathic pain, in a mammal in need thereof, comprising administering a therapeutically effective amount of T-62 crystals as obtainable by the methods of the present invention, in particular Form I crystals of T-62.

The term “treatment” shall be understood as the management and care of a patient for the purpose of combating the disease, condition or disorder, e.g., pain management.

In carrying out the methods of the present invention, crystal forms of T-62 may be formulated into pharmaceutical compositions suitable for administration via a variety of routes, such as oral or rectal, transdermal, intrathecal and parenteral administration to mammals, including man. For oral administration the pharmaceutical composition comprising T-62 can take the form of solutions, suspensions, tablets, pills, capsules, powders, microemulsions, unit dose packets and the like. Preferred are tablets and gelatin capsules comprising crystals of T-62 together with: a) diluents, e.g., lactose, dextrose, sucrose, mannitol, sorbitol, cellulose, glycine and/or vegetable oils; b) lubricants, e.g., silica, talcum, stearic acid, its magnesium or calcium salt, polyethylene glycols and glycerol esters; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) surfactants, absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-90%, preferably about 1-80%, of the active ingredient(s), conveniently from 30-95% for tablets and capsules, and 3-50% for liquid preparations.

The dosage of T-62, in any of its forms, can depend on a variety of factors, such as mode of administration, homeothermic species, age and/or individual condition.

In general, the doses of T-62 to be administered to warm-blooded animals, including man, of approximately 75 kg body weight, especially the doses effective for enhancing the adenosine A₁ receptor function, e.g., to alleviate pain, are from about 1 mg to about 1 g, preferably from about 10 mg to about 500 mg. Two per day are an option, one for daytime use and one for nighttime use. Since there is the potential of an allosteric adenosine A₁ receptor enhancer to cause sedation at a high dose, the higher doses are recommended for night use. For example, a dose of from about 100 to about 500 mg dose of T-62 in tablet form is recommended for daytime use while a dose from about 600 to about 1000 mg is recommended as a nighttime dose.

The efficacy of T-62 may be assessed using, e.g., pain models such as carrageenan model (Guilbaud and Kayser, Pain 1987, 28, 99-107) for acute inflammatory pain, FCA model (Freund's Complete Adjuvant; Hay et al., Neuroscience 1997, 78(3), 843-850) for chronic inflammatory pain, CCl model (Chronic Constriction Injury; Bennett and Xie, Pain 1988, 33, 87-107) for neuropathic pain, or postincisional hypersensitivity model (Obata et al., Anesthesiology 2004, 100, 1258-1262) for postoperative pain.

The present invention is further described by the following examples. The examples are provided solely to illustrate the invention by reference to specific embodiments. These examples, while illustrating certain specific aspects of the invention, do not portray the limitations or circumscribe the scope of the disclosed invention.

### EXAMPLE 1

### A Process for Preparing Form I Crystals of T-62

A solution of 2.0 g of T-62 (Form I crystals) in 8 mL of 95% ethanol (EtOH:water-95:5) is heated at 78° C. with stirring for about 30 min to achieve a complete dissolution. The solution is then cooled to either 4° C. or 30° C. employing different cooling rates and continuous stirring. After the solution is cooled to the desired temperature, the precipitated solids are collected by vacuum filtration. The final crystalline form, yield, cooling rates, particle morphology and particle size of each crystallization batch are examined. In each case, Form I crystals are produced. A slightly better yield is observed for the 4° C. crystallization in general, since lower temperatures tend to yield more solids. Crash cooling yields solids that are easier to filter (takes about 5 min to filter), whereas slow cooling yields solids that are more difficult to filter (takes about 30 min to filter). The crash cooled crystals exhibit narrow particle size distribution, whereas the slow cooled crystals exhibit larger particle size distribution. The particle size data are consistent with the optical microscopy data. Therefore, if narrower particle distribution is desired, the optimal crystallization would be cooling the solution from 78GC to 4° C. quickly (for 9.0 g scale about 5 min). The results are summarized in Table 6 below.

TABLE 6

| | Yield | |
|---|---|---|
| Method | (%) | Observation |
| Heated to 78° C., held for ~30 min; | ~90 | ~5 min to filter solution; |
| crash cooled in ice/water bath to | | solids appear at ~65° C. |
| 4° C. in 5 min (CC) | | |
| Heated to 78° C., held for ~30 min; | ~91 | ~15 min to filter solution; |
| cooled to 4° C. in ~156 min (SC) | | solids appear at ~67° C. |
| Heated to 78° C., held for ~30 min; | ~91 | ~5 min to filter solution; |
| crash cooled in ambient water | | solids appear at ~62° C. |
| bath to 30° C. in 2 min (CC) | | |
| Heated to 75° C., held for ~30 min; | ~83 | ~30 min to filter solution; |
| cooled to 30° C. in ~49 min (SC) | | solids appear at ~71° C. |
| Heated to 78° C., held for ~30 min; | ~86 | ~5 min to filter solution; |
| crash cooled in ambient water | | solids appear at ~58° C. |
| bath to 30° C. in 2 min (CC) | | |

### EXAMPLE 2

### A Process for Preparing Form II Crystals of T-62

A. 67.8 mg of T-62 (Form I crystals) is dissolved in 5 mL of ethyl acetate at room temperature. The solution is filtered through a 0.2-μm nylon syringe filter into an amber 20-mL scintillation vial. The sample vial is placed, uncapped, in a dark fume hood and allowed to evaporate to dryness to afford Form II crystals of T-62.

B. 49.6 mg of T-62 (Form I crystals) is dissolved in 4.5 mL of methanol with sonication at room temperature. The solution is filtered through a 0.2-μm nylon syringe filter into an amber 20-mL scintillation vial. The sample vial is placed, uncapped, in a dark fume hood and allowed to evaporate dryness to afford Form II crystals of T-62.

### EXAMPLE 3

### A Process for Preparing Form III Crystals of T-62

A. 15 mL of Hexanes is chilled in a dry ice/acetone bath for approximately 0.5 h. 150.3 mg of T-62 (Form I crystals) is dissolved in 2.0 mL of dioxane at room temperature. The resulting solution is filtered through a 0.2-μm nylon syringe filter into the chilled hexanes. Solids are formed immediately. The solids are collected by vacuum filtration, transferred into an amber vial, and capped to afford Form III crystals of T-62.

B. 511.8 mg of T-62 (Form I crystals) is dissolved in 6.6 mL of dioxane with sonication at room temperature. The resulting solution is filtered through a 0.2-μm nylon syringe filter into a vessel containing 50 mL of hexanes chilled by immersion in a dry ice/isopropanol bath for approximately 0.5 h. Solids are formed immediately. The solids are collected by vacuum filtration, transferred into a 20-mL scintillation vial and dried at an ambient temperature under vacuum overnight to afford Form III crystals of T-62.

### EXAMPLE 4

### A Process for Preparing Form IV Crystals of T-62

3-mL of Isopropanol is heated to 62° C. The heated solvent is added to 163.8 mg of T-62 (Form I crystals) while stirring on a hotplate at approximately 60° C. When all the solids are dissolved, a spatula-tip-full of T-62 is added. Solids persist. The sample is filtered into a clean 20-mL scintillation vial. This solution is left on the hotplate, which is then turned off to allow slow cooling. After 1 day, the sample is moved to a refrigerator, and 2 days later moved to a freezer. After 20 days, the liquid portion of the sample is decanted into an amber 20-mL scintillation vial. The vial is covered with aluminum foil, which is punctured with 5 pinholes, to allow for slow evaporation to dryness to afford Form IV crystals of T-62.

### EXAMPLE 5

### Approximate Solubilities of Form I Crystals of T-62

Solubilities for Form I crystals of T-62 may be estimated based on total solvent used to give a clear solution. A test solvent, either reagent or HPLC (high pressure liquid chromatography) grade, is added in measured aliquots, e.g., 200 or 500 μL, to an accurately weighed sample of T-62 (typically 50 to 90 mg). For some samples, sonication is applied between solvent additions to aid dissolution. Generally, solvent is added until a clear solution is obtained. Actual solubilities may be greater than those shown in Table 7 because of the volume of the solvent portions utilized or a slow rate of dissolution.

TABLE 7

| Solvent | Solubility (mg/mL, RT) |
|---|---|
| acetone | 59.8 |
| ACN | 11.1 |
| CHCl₃ | 174.6 |
| dichloromethane | 85.3 |
| dioxane | 74.9 |
| N,N-dimethylformamide (DMF) | 128.6 |
| dimethylsulfoxide (DMSO) | 182.8 |
| EtOH | 16.6 |
| EtOAc | 67.8 |
| diisopropyl ether | 5.9 |
| octanol | 19.7 |
| hexanes | 5.6 |
| MeOH | 19.8 |
| MEK | 132.2 |
| nitromethane | 12.0 |
| THF | 338.5 |
| toluene | 45.7 |
| water | practically insoluble |
| IPA | 13.1 |
| di-n-butyl ether | 21.4 |
| cyclohexane | 9.8 |
| ACN:H₂O - 1:1 | 8.4 |
| MeOH:H₂O - 95:5 | 8.7 |
| EtOH:H₂O - 95:5 | 12.8 |

### EXAMPLE 6

### Formulation A

T-62 may be obtained from King Pharmaceuticals (Cary, N.C.) in dry powder form. T-62 is screened through a #40 screen and then added to a mixture of propylene glycol monocaprylate (Capryol 90®), caprylocaproyl macrogol-8 glycerides (Labrasol®), super refined soybean oil (USP) and polysorbate 80 (Crillet 4 HP®) at 50° C. (±5° C.) while mixing with a propeller mixer to dissolve T-62. The mixture/solution is sparged with nitrogen throughout the process. The resulting solution is then pumped though a 5 μm Meissner filter capsule, and had a density of 1.006 g/mL at 25° C. The solution may then be encapsulated into soft elastic gelatin capsules (Capsugel, Inc.) to afford a dose of 30 mg/mL (Table 8).

TABLE 8

| Ingredient | w-% |
|---|---|
| T-62 | 6.08 |
| propylene glycol monocaprylate (Capryol 90 ®) | 43.92 |
| caprylocaproyl macrogol-8 glycerides (Labrasol ®) | 16.70 |
| super refined soybean oil (USP) | 25.00 |
| polysorbate 80 (Crillet 4 HP ®) | 8.30 |
| TOTAL | 100 |

### EXAMPLE 7

### Formulation B

T-62 (dry powder) is milled using a Quadro Comil 197 with screen (2A018R01530) and impeller (2A16011730212) at 2400 rpm. The milled T-62 is then added to a mixture of super refined soybean oil (USP), propylene glycol monocaprylate (Capryol 90®), caprylocaproyl macrogol-8 glycerides (Labrasol®), and polysorbate 80 (Crillet 4 HP®) at 50-55° C. The mixture/solution is sparged with nitrogen throughout the process. The mixture is stirred until T-62 dissolved, then pumped though a 5 μm Meissner filter capsule. The resulting solution may then be encapsulated into hard gelatin capsules (Capsugel) to afford a dose of 70 mg/mL (Table 9).

TABLE 9

| Ingredient | w-% |
|---|---|
| T-62 | 8.33 |
| propylene glycol monocaprylate (Capryol 90 ®) | 41.67 |
| caprylocaproyl macrogol-8 glycerides (Labrasol ®) | 16.70 |
| super refined soybean oil (USP) | 25.00 |
| polysorbate 80 (Crillet 4 HP ®) | 8.30 |
| TOTAL | 100 |

### EXAMPLE 8

### Formulation C

T-62 (dry powder) is milled using a Quadro Comil 197 with screen (2A018R01530) and impeller (2A16011730212) at 2400 rpm. The milled T-62 is then micronized using a Glen Mills Jet Mill with nitrogen as the propellant. T-62 is passed through the Jet Mill twice to reduce the particle size to a mean diameter of 12.2 μm. The micronized T-62 is added to a mixture of super refined soybean oil (USP) with propylene glycol monocaprylate (Capryol 90®), caprylocaproyl macrogol-8 glycerides (Labrasol®), and polysorbate 80 (Crillet 4 HP®) at 50-55° C. using a propeller type mixer to dissolve T-62. The mixture/solution is sparged with nitrogen throughout the process. The resulting solution is then pumped though a 5 μm Meissner filter capsule, and may then be encapsulated into hard gelatin capsules (size 00 Capsules, obtained from Capsugel®) to afford a dose of 70 mg/mL (Table 10).

TABLE 10

| Ingredient | w-% |
|---|---|
| T-62 | 8.33 |
| propylene glycol monocaprylate (Capryol 90 ®) | 41.67 |
| caprylocaproyl macrogol-8 glycerides (Labrasol ®) | 16.70 |
| super refined soybean oil (USP) | 25.00 |
| polysorbate 80 (Crillet 4 HP ®) | 8.30 |
| TOTAL | 100 |

### EXAMPLE 8

### Formulation D

T-62 (dry powder) is milled using a Quadro Comil 197 with screen (2A018R01530) and impeller (2A16011730212) at 2400 rpm. The milled T-62 is then added to a mixture of propylene glycol monocaprylate (Capryol 90®), caprylocaproyl macrogol-8 glycerides (Labrasol®), and polysorbate 80 (Crillet 4 HP®) at 45±5° C. using a propeller type mixer to dissolve T-62. The mixture/solution is sparged with nitrogen throughout the process. Super refined soybean oil (USP) is then added with continued mixing. The solution is allowed to return to room temperature, and then pumped though a 5 μm Meissner filter capsule. The resulting solution may then be encapsulated into soft elastic gelatin capsules (Capsugel, Inc.) to afford a dose of 100 mg/mL (Table 11).

TABLE 11

| Ingredient | w-% |
|---|---|
| T-62 | 8.33 |
| propylene glycol monocaprylate (Capryol 90 ®) | 41.67 |
| caprylocaproyl macrogol-8 glycerides (Labrasol ®) | 29.20 |
| super refined soybean oil (USP) | 12.50 |
| polysorbate 80 (Crillet 4 HP ®) | 8.30 |
| TOTAL | 100 |

### EXAMPLE 8

### Formulation E

T-62 (dry powder) is milled using a Quadro Comil 197 with screen (2A018R01530) and impeller (2A16011730212) at 2400 rpm. The milled T-62 is then added to a mixture of super refined soybean oil (USP) with propylene glycol monocaprylate (Capryol 90®), caprylocaproyl macrogol-8 glycerides (Labrasol®), polysorbate 80 (Crillet 4 HP®) and ethanol at 45±5° C. using a propeller type mixer to dissolve T-62. The mixture/solution is sparged with nitrogen throughout the process. Super refined soybean oil is then added with continued mixing. The solution is allowed to return to room temperature, and then pumped though a 5 μm Meissner filter capsule. The resulting solution may then be encapsulated into soft elastic gelatin capsules (size 00 Capsules, obtained from Capsugel®) to afford a dose of 100 mg/mL (Table 12).

TABLE 12

| Ingredient | w-% |
|---|---|
| T-62 | 8.33 |
| propylene glycol monocaprylate (Capryol 90 ®) | 41.67 |
| caprylocaproyl macrogol-8 glycerides (Labrasol ®) | 21.20 |
| super refined soybean oil (USP) | 12.50 |
| polysorbate 80 (Crillet 4 HP ®) | 8.30 |
| ethanol | 8.00 |
| TOTAL | 100 |

What is claimed is:

1. A crystal form of anhydrous (2-amino-4,5,6,7-tetrahydrobenzo[b]thiophen-3-yl)(4-chlorophenyl)methanone (T-62) which crystal form (Form I) is substantially free of other polymorphic forms of T-62 and has at least one of the following properties:
(a) a melting point of about 143° C.;
(b) a X-ray diffraction pattern with characteristic X-ray diffraction peaks at diffraction angles (20) of about 19.6±0.2, 19.9±0.2, 20.8±0.2, 22.9±0.2 and 23.3±0.2;
(c) an infrared absorption spectrum with absorption bands at about 3330±2, 3239±2, 2943±2, 1908±2 and 927±2 cm⁻¹; and
(d) a Raman spectrum with Raman shifts at about 3060±2, 2944±2, 1596±2, 1426±2 and 1391±2 cm⁻¹.

2. A crystal form according to claim 1, which crystal form has characteristic X-ray diffraction peaks at diffraction angles (20), and relative intensities (I/I₁) of about:

| Angle (deg 2θ) | Relative intensity (I/I₁) |
|---|---|
| 11.9 ± 0.2 | 11 |
| 13.3 ± 0.2 | 13 |
| 13.9 ± 0.2 | 11 |
| 18.6 ± 0.2 | 14 |
| 19.6 ± 0.2 | 100 |
| 19.9 ± 0.2 | 40 |
| 20.8 ± 0.2 | 48 |
| 22.9 ± 0.2 | 37 |
| 23.3 ± 0.2 | 46 |
| 24.0 ± 0.2 | 22 |
| 25.7 ± 0.2 | 18 |
| 26.7 ± 0.2 | 20 |
| 27.6 ± 0.2 | 13 |
| 29.2 ± 0.2 | 23 |
| 38.2 ± 0.2 | 10 |

3. A crystal form according to claim 1, which crystal form has all four of the properties (a), (b), (c) and (d).

4. A pharmaceutical composition comprising a therapeutically effective amount of a crystal form according to claim 1 and a pharmaceutically acceptable carrier.

5. A crystal form of anhydrous (2-amino-4,5,6,7-tetrahydrobenzo[b]thiophen-3-yl)(4-chlorophenyl)methanone (T-62) which crystal form (Form II) is substantially free of other polymorphic forms of T-62 and has at least one of the following properties:
(a) a melting point of about 135° C.;
(b) a X-ray diffraction pattern with characteristic X-ray diffraction peaks at diffraction angles (2θ) of about 16.5±0.2, 19.4±0.2, 20.3±0.2, 22.2±0.2, 23.6±0.2 and 26.2±0.2;
(c) an infrared absorption spectrum with absorption bands at about 3344±2, 3243±2, 2955±2, 2930±2, 1895±2 and 1172±2 cm⁻¹; and
(d) a Raman spectrum with Raman shifts at about 3065±2, 2957±2, 1595±2, 1430±2 and 1394±2 cm⁻¹.

6. A crystal form according to claim 5, which crystal form has characteristic X-ray diffraction peaks at diffraction angles (2θ), and relative intensities (I/I₁) of about:

| Angle (deg 2θ) | Relative intensity (I/I₁) |
|---|---|
| 6.7 ± 0.2 | 26 |
| 11.6 ± 0.2 | 24 |
| 13.5 ± 0.2 | 17 |
| 15.5 ± 0.2 | 10 |
| 16.5 ± 0.2 | 100 |
| 19.4 ± 0.2 | 52 |
| 20.3 ± 0.2 | 68 |
| 21.1 ± 0.2 | 24 |
| 21.6 ± 0.2 | 28 |
| 22.2 ± 0.2 | 37 |
| 22.8 ± 0.2 | 24 |
| 23.6 ± 0.2 | 40 |
| 24.6 ± 0.2 | 27 |
| 26.2 ± 0.2 | 34 |
| 28.5 ± 0.2 | 12 |
| 29.0 ± 0.2 | 18 |
| 30.0 ± 0.2 | 13 |
| 30.9 ± 0.2 | 24 |
| 33.4 ± 0.2 | 14 |
| 35.4 ± 0.2 | 20 |
| 37.5 ± 0.2 | 10 |
| 39.0 ± 0.2 | 19 |

7. A crystal form according to claim 5, which crystal form has all four of the properties (a), (b), (c) and (d).

8. A pharmaceutical composition comprising a therapeutically effective amount of a crystal form according to claim 5 and a pharmaceutically acceptable carrier.

9. A crystal form of (2-amino-4,5,6,7-tetrahydrobenzo[b]thiophen-3-yl)(4-chlorophenyl)methanone (T-62) which crystal form (Form III) is substantially free of other polymorphic forms of T-62 and has at least one of the following properties:
(a) a melting point in the range of about 126-135° C.;
(b) a X-ray diffraction pattern with characteristic X-ray diffraction peaks at diffraction angles (20) of about 15.5±0.2, 19.1±0.2, 19.4±0.2, 21.3±0.2 and 21.7±0.2;
(c) an infrared absorption spectrum with absorption bands at about 3247±2, 2941±2, 1908±2 and 922±2 cm⁻¹; and
(d) a Raman spectrum with Raman shifts at about 3068±2, 2939±2, 1596±2 and 1436±2 cm⁻¹.

10. A crystal form according to claim 9, which crystal form has characteristic X-ray diffraction peaks at diffraction angles (2θ), and relative intensities (I/I₁) of about:

| Angle (deg 2θ) | Relative intensity (I/I₁) |
|---|---|
| 15.5 ± 0.2 | 40 |
| 16.9 ± 0.2 | 12 |
| 19.1 ± 0.2 | 57 |
| 19.4 ± 0.2 | 34 |
| 21.3 ± 0.2 | 34 |
| 21.7 ± 0.2 | 100 |
| 22.9 ± 0.2 | 21 |
| 24.6 ± 0.2 | 12 |
| 29.4 ± 0.2 | 19 |
| 30.5 ± 0.2 | 18 |

11. A crystal form according to claim 9, which crystal form has all four of the properties (a), (b), (c) and (d).

12. A pharmaceutical composition comprising a therapeutically effective amount of a crystal form according to claim 9 and a pharmaceutically acceptable carrier.

13. A crystal form of (2-amino-4,5,6,7-tetrahydrobenzo[b]thiophen-3-yl)(4-chlorophenyl)methanone (T-62) which crystal form (Form IV) is substantially free of other polymorphic forms of T-62 and has at least one of the following properties:
(a) a melting point of about 138° C.; and
(b) a X-ray diffraction pattern with characteristic X-ray diffraction peaks at diffraction angles (2θ) of about 7.4±0.2, 18.8±0.2, 20.4±0.2 and 21.3±0.2.

14. A crystal form according to claim 13, which crystal form has characteristic X-ray diffraction peaks at diffraction angles (2θ), and relative intensities (I/I₁) of about:

| Angle (deg 2θ) | Relative intensity (I/I₁) |
|---|---|
| 7.4 ± 0.2 | 45 |
| 8.8 ± 0.2 | 15 |
| 13.2 ± 0.2 | 10 |
| 13.6 ± 0.2 | 21 |
| 16.1 ± 0.2 | 25 |
| 18.4 ± 0.2 | 11 |
| 18.8 ± 0.2 | 43 |
| 19.5 ± 0.2 | 21 |
| 20.4 ± 0.2 | 38 |
| 21.3 ± 0.2 | 100 |
| 22.1 ± 0.2 | 10 |
| 23.2 ± 0.2 | 25 |
| 24.5 ± 0.2 | 20 |
| 25.6 ± 0.2 | 15 |
| 26.8 ± 0.2 | 13 |
| 27.8 ± 0.2 | 24 |

15. A crystal form according to claim 13, which crystal form has both the properties (a) and (b).

16. A pharmaceutical composition comprising a therapeutically effective amount of a crystal form according to claim 13 and a pharmaceutically acceptable carrier.

17. A method for the production of Form I crystal form of (2-amino-4,5,6,7-tetrahydrobenzo[b]thiophen-3-yl)(4-chlorophenyl)methanone (T-62) which crystal form is substantially free of other polymorphic forms of T-62, wherein the method comprises:
(a) dissolving T-62, in any of its forms, in a solvent to form a solution in which solvent T-62 is suitably soluble at an elevated temperature ranging from about 30° C. to the boiling point of the solvent, and in which solvent T-62 is only poorly soluble at a lower temperature ranging from about −78° C. to about 30° C.;
(b) cooling the solution to the lower temperature to induce precipitation of the Form I crystal form of T-62; and
(c) isolating and drying the precipitated Form I crystal form of T-62.

18. The method of claim 17, wherein the solvent is a mixture of a lower alcohol and water.

19. The method of claim 18, wherein the lower alcohol is ethanol.

20. The method of claim 19, wherein the solvent contains about 95% of ethanol by volume.

21. The method of claim 20, wherein the elevated temperature ranges from about 60° C. to about 80° C.; and the lower temperature ranges from about 4° C. to about 30° C.

22. The method of claim 21, wherein the elevated temperature is about 78° C.

23. A method for the production of Form I crystal form of (2-amino-4,5,6,7-tetrahydrobenzo[b]thiophen-3-yl)(4-chlorophenyl)methanone (T-62) which crystal form is substantially free of other polymorphic forms of T-62, wherein the method comprises:
(a) dissolving T-62, in any of its forms, in a first solvent to form a solution in which solvent T-62 is readily soluble at a temperature ranging from room temperature to the boiling point of the solvent;
(b) treating the solution with a second solvent which is miscible with the first solvent, and in which T-62 is only poorly soluble to induce precipitation of Form I crystal form of T-62;
(c) if desired, cooling the resulting mixture gradually to a temperature of about 10° C. to further induce precipitation of the Form I crystal form of T-62; and
(d) isolating and drying the precipitated Form I crystal form of T-62.

24. The method of claim 23, wherein the first solvent is selected from the group consisting of acetone, methanol, isopropanol, methylethylketone and tetrahydrofuran.

25. The method of claim 23, wherein the second solvent is selected from the group consisting of hexane and water.

26. The method of claim 23, wherein the first solvent is selected from the group consisting of acetone, methylethylketone and tetrahydrofuran; and the second solvent is hexane.

27. The method of claim 23, wherein the first solvent is selected from the group consisting of methanol, methylethylketone and tetrahydrofuran; and the second solvent is water.

28. The method of claim 23, wherein the first solvent is isopropanol; and the second solvent is water.

29. A method for the production of Form I crystal form of (2-amino-4,5,6,7-tetrahydrobenzo[b]thiophen-3-yl)(4-chlorophenyl)methanone (T-62) which crystal form is substantially free of other polymorphic forms of T-62, wherein the method comprises converting the Form III or Form IV crystal form of T-62 into the Form I crystal form of T-62 by storing the former crystal form of T-62 under atmospheric, super-atmospheric or reduced pressure at a temperature ranging from about 20° C. to about 104° C. and in relative humidity ranging from about 0% to about 95%.
